(19) United States
(12) Patent Application Publication
(10) Pub. No.: US 2011/0172647 A1
(43) Pub. Date: Jul. 14, 2011

(54) SURGICAL INSTRUMENT

(75) Inventors: MARTIN WENZLER, Balgheim (DE); Gebhard Wenzler, Balgheim (DE)

(73) Assignee: RAIMUND WENZLER GMBH, Balgheim (DE)

(21) Appl. No.: 12/956,683

(22) Filed: Nov. 30, 2010

(30) Foreign Application Priority Data
Nov. 30, 2009 (DE) 10 2009 056 099.8

(51) Int. Cl. A61B 17/00

(52) U.S. Cl. 606/1

(57) ABSTRACT

The invention concerns a surgical instrument with a base part, which has a shaft and a stationary handle branch. A working part is guided movably on the shaft and a swiveling handle branch is arranged on the base part, which is mounted able to swivel in an opening of the base part in order to activate the working part by means of a pivot pin. The pivot pin engages with a through opening in the swiveling handle branch, and for the purpose of separating the swiveling handle branch from the working part, the pivot pin can be entirely lifted out axially from the swiveling handle branch and is captively secured to the base part. The swiveling handle branch in the position separated from the working part is captively secured to the base part.

### BACKGROUND OF THE INVENTION

1. Field of the Invention

The present invention relates to a surgical instrument with a base part, which has a shaft and a stationary handle branch. More specifically, the present invention relates to a surgical instrument which can be easily manipulated for cleaning, yet have the individual parts of the surgical instrument stay with each other.

2. Description of the Related Art

The related art involves with a base part, having a shaft and a stationary handle branch, wherein a working part is movably guided on the shaft, and wherein a swivelable handle branch is disposed on the base part, being mounted and able to swivel in an opening of the base part in order to activate the working part by means of a pivot pin, engaging in a through opening of the swivelable handle branch. In most such surgical instruments, the pivot pin and the swiveling handle branch are firmly joined together. DE 198 19 382 C1 shows one such surgical instrument, in which the pivot pin can be axially lifted out entirely from the swiveling handle branch in order to separate the swiveling handle branch from the working part and wherein it is captively held on the base part, thereby making it possible to draw the handle branch entirely out of the opening in the base part and release the working part for detachment from the base part, so that the surgical instrument can be fully taken apart in this way.

However, it is also desirable to make it possible to take apart the surgical instrument for cleaning purposes, yet have the individual parts of the surgical instrument stay with each other. Therefore, in many surgical instruments a loosening of the working part from the base part is possible, while the working part remains fixed to the swiveling handle branch.

What is not appreciated by the prior art is the lack of comfort and ease of use associated with manipulating a surgical instrument of this kind.

Accordingly, there is a need for an improved surgical instrument which can be easily manipulated for cleaning, yet have the individual parts of the surgical instrument stay with each other.

### ASPECTS AND SUMMARY OF THE INVENTION

An aspect of the present invention is to provide a an improved surgical instrument which can be easily manipulated for cleaning, yet have the individual parts of the surgical instrument stay with each other.

The surgical instrument according to the invention with a base part, which has a shaft and a stationary handle branch, wherein a working part is guided movably on the shaft and wherein a swiveling handle branch is arranged on the base part, which is mounted able to swivel in an opening of the base part in order to activate the working part by means of a pivot pin, which engages with a through opening in the swiveling handle branch, and wherein for the purpose of separating the swiveling handle branch from the working part the pivot pin can be entirely lifted out axially from the swiveling handle branch and is captively secured to the base part, is characterized in that the swiveling handle branch in the position separated from the working part is captively secured to the base part.

In this way, the invention on the one hand enables a separation between handle branch and working part in that the handle branch can be pulled at least partly out from the base part, but on the other hand it makes it possible to fix in place both the handle branch and also, if so desired, the working part captively and independently of each other on the base part, so as to enable an especially thorough cleaning of the surgical instrument, while at all times all the components of the surgical instrument are joined to each other so that the user, after the cleaning, does not have to look for any of the individual components once again, but simply puts the surgical instrument back together.

In a first preferred embodiment of the present invention, a groove is arranged in one lateral surface of the swiveling handle branch, starting from the through opening, with which the pivot pin engages in the position of the swiveling handle branch separated from the working part. The groove thus forms a guide for the movement of the handle branch, especially during partial removal of the handle branch from the base part, while the end of the groove away from the through opening forms an end stop for the pivot pin, so that the swiveling handle branch is prevented from being removed entirely from the opening in the base part. In this position, while the handle branch releases the working part to enable a detachment of the working part from the base part, it nonetheless remains captively secured to the base part.

In an alternative preferred embodiment of the present invention, a second through opening is arranged in the swiveling handle branch, with which the pivot pin engages in the position of the swiveling handle branch separated from the working part. Also in this way the aforementioned benefits of the captive handle branch are achieved, while a second through opening can make possible a more stable support between the pivot pin and the swiveling handle branch.

According to one advantageous development of the present invention, the through opening and the second through opening are joined by means of a groove on one lateral surface of the swiveling handle branch, so that when the swiveling handle branch moves out from the opening in the base part the pivot pin is guided in the groove and taken in particular to the second through opening, which makes it easier for the user to pull out the swiveling handle branch and push it back in after the cleaning.

Preferably, the pivot pin is connected via a yoke to a release bolt, which is arranged axially parallel to the pivot pin and able to move axially in the base part outside the swivel range of the swiveling handle branch. This makes it possible to mount the release bolt as well so that it can move axially in the base part, with the release bolt passing entirely through the base part in the transverse direction. In particular, this makes it possible to pull the pivot pin out from the opening and out from the through opening of the swiveling handle branch by axial pressure on the release bolt, making possible an especially simple activation.

According to another preferred embodiment of the invention, the swiveling handle branch engages by a rounded head in a recess of the working part. The benefit of the rounded head of the swiveling handle branch is that this is very stable and a robust, swiveling connection between the swiveling handle branch and the working part can be produced in easy fashion. It is especially preferred to configure the recess in the manner of a step bearing for this head, so that the head is automatically positioned and oriented in the recess of the working part when the swiveling handle branch is inserted, which additionally facilitates the putting of the instrument together.

Preferably the recess of the working part is formed as an opening through the working part. On the one hand, an opening can be better cleaned than a pocketlike recess. On the other hand, the working part can be configured especially flat in this way, in order to provide the user with good visibility along the working part up to the distal working end of the surgical instrument.

Advantageously, the working part is arranged able to swivel on the base part in the position of the swiveling handle branch separated from the working part, so as to secure the working part captively to the surgical instrument in this way and keep all components of the surgical instrument together, even when cleaning the surgical instrument.

According to a preferred embodiment of the invention, the base part has an oblong hole passing through its proximal end and the working part is guided movably in the oblong hole and mounted so that it can swivel, so as to make possible in this way the most simple possible fastening of the working part to the base part and at the same time not hinder the movement of the working part in the assembled state of the surgical instrument relative to the shaft. The oblong hole furthermore offers the benefit of a very robust guideway, which cannot seize up even with poor lubrication.

Preferably, the base part has a smaller width in the region adjoining the oblong hole and on the working part there are arranged two cheeks extending laterally on the base part, which are joined to a pin that passes through the oblong hole. In this way, a fastening of the working part to the oblong hole is accomplished in simple manner, while on the other hand the width of the surgical instrument is not increased in this way.

The above, and other aspects, features and advantages of the present invention will become apparent from the following description read in conjunction with the accompanying drawings, in which like reference numerals designate the same elements.

### BRIEF DESCRIPTION OF THE DRAWINGS

FIG. 1A is a side view of a first sample embodiment of the surgical instrument of the present invention.

FIG. 1B is a partially sectioned top view of the surgical instrument of FIG. 1A.

FIG. 1C is a rear view of the surgical instrument per FIG. 1A.

FIG. 1D is another side view of the surgical instrument per FIG. 1A.

FIG. 2A is a view of the surgical instrument per FIG. 1A in a cleaning position.

FIG. 2B is another side view of the surgical instrument per FIG. 2A in the cleaning position.

FIG. 3A is an exploded view of the surgical instrument per FIG. 1A.

FIG. 3B is a perspective representation of the swiveling handle branch of the surgical instrument per FIG. 1A.

FIG. 3C is a side view of the handle branch per FIG. 3B.

FIG. 3D is another side view of the handle branch per FIG. 3B.

FIG. 3E is another side view of the handle branch per FIG. 3B.

FIG. 3F is an enlarged cutout feature of FIG. 3D.

FIG. 3G is a longitudinal section through the head of the handle branch per FIG. 3F.

FIG. 4A is a side view of a second sample embodiment of a surgical instrument.

FIG. 4B is an enlarged cutout feature of FIG. 4A.

FIG. 4C is a section along line A-A in FIG. 4B.

FIG. 4D is a section along line A-A in FIG. 4B with a depressed release button.

FIG. 4E is the surgical instrument per FIG. 4A in a cleaning position.

FIG. 4F is the base part of the surgical instrument per FIG. 4A in a side view.

FIG. 4G is a top view of the base part per FIG. 4F.

FIG. 4H is a cross sectional view as seen along line A-A in FIG. 4F.

FIG. 4I is an enlarged cutout feature as shown at X of FIG. 4G.

FIG. 4J is a top view of the working part of the surgical instrument per FIG. 4A.

FIG. 4K is a partly sectional side view of the working part per FIG. 4J.

FIG. 4L is a section through the working part along line A-A in FIG. 4K.

FIG. 4M is a side view of the swiveling handle branch of the surgical instrument per FIG. 4A.

FIG. 4N is another side view of the handle branch per FIG. 4M.

FIG. 4O is another side view of the handle branch per FIG. 4M.

### DETAILED DESCRIPTION OF THE PREFERRED EMBODIMENTS

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, and below may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices.

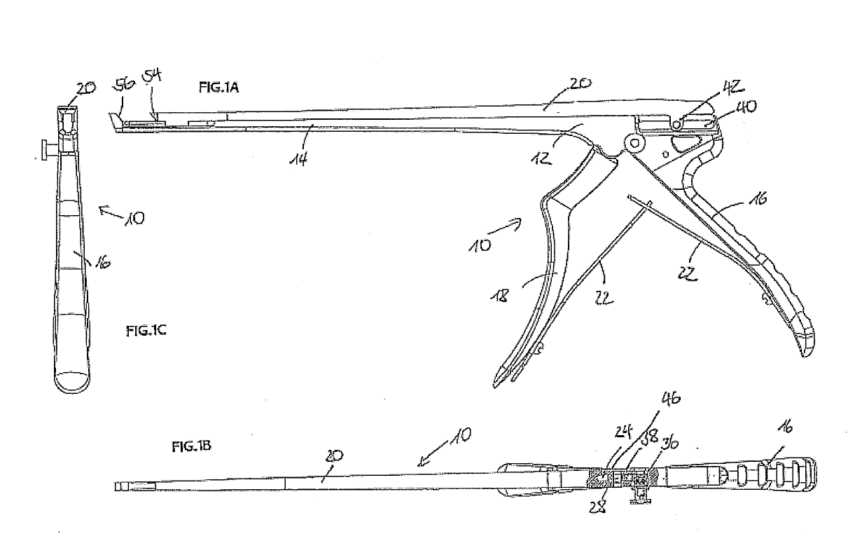

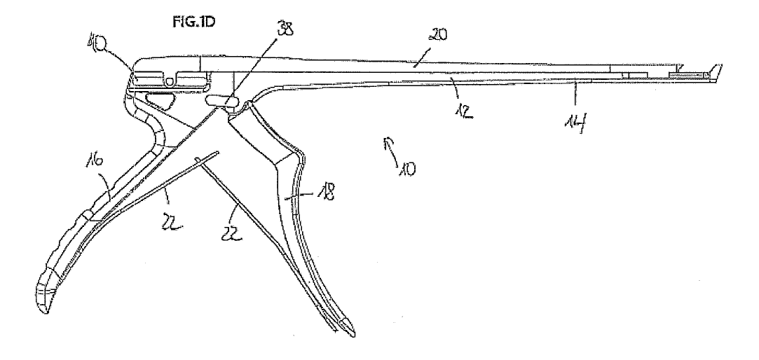

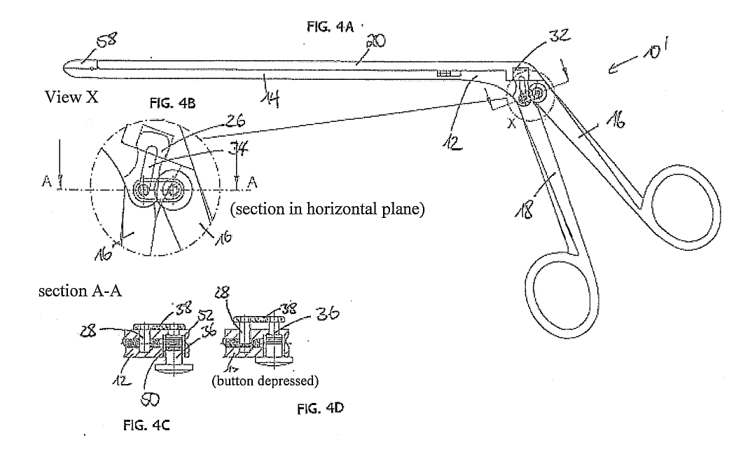

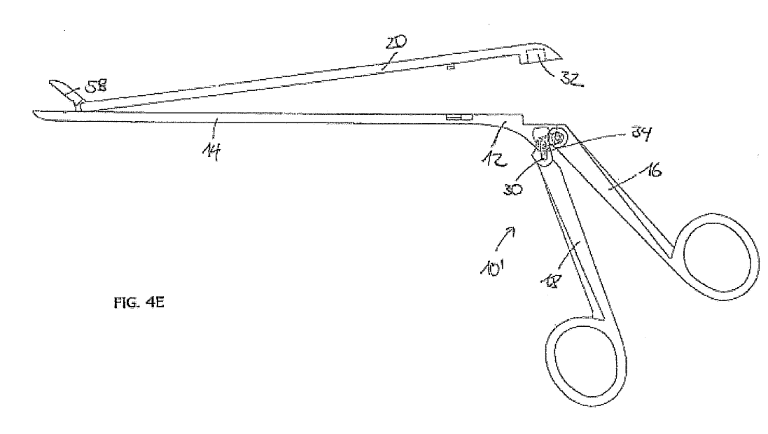

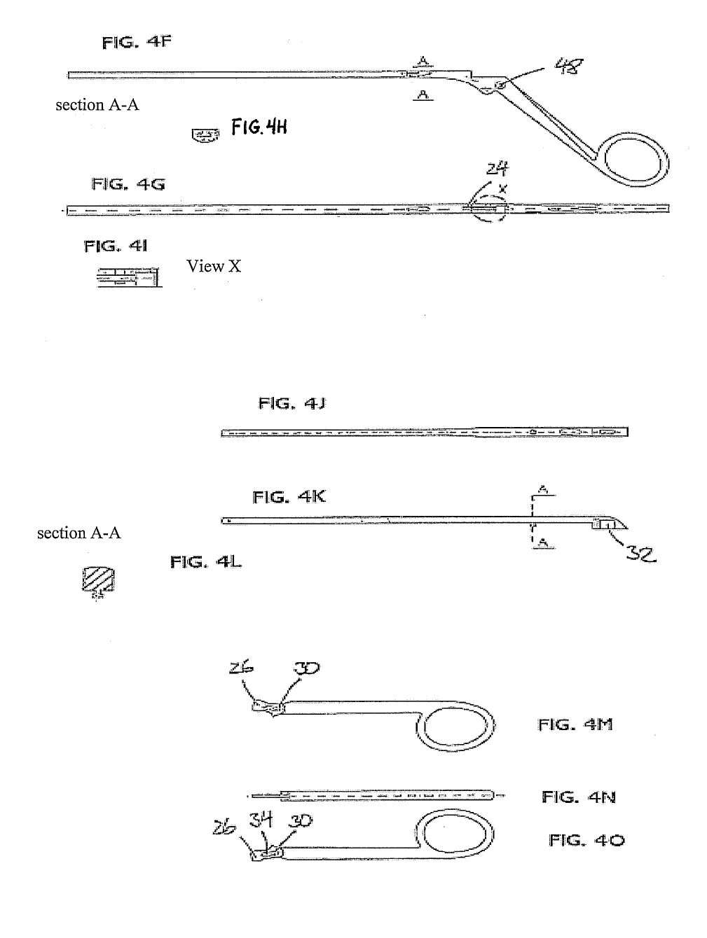

FIGS. 1, 2 and 3 show different views of a first sample embodiment of a surgical instrument 10, while FIGS. 4A to 4O show different views of a second sample embodiment of a surgical instrument 10′. Since a lot of the components of the two surgical instruments 10, 10′ have identical functions, the same components in the figures are designated by the same reference numbers, while for better clarity all reference numbers are indicated in each figure.

The surgical instruments 10, 10′ are both configured as sliding shaft instruments and have a base part 12, which has a shaft 14 and fixed handle branch 16. On the shaft 14 is guided a working part 20 that can move in the lengthwise direction of the shaft 14. At the proximal end of the shaft 14, the base part 12 passes into the fixed handle branch 16 as a single piece. A swiveling handle branch 18 is mounted on the base part 12 and engages in the working part 20 in a way described more fully afterwards, so that by swiveling the swiveling handle branch 18 against the fixed handle branch 16 the working part 20 is moved on the shaft 14 in order to activate the instrument 10, 10′.

The instrument 10 is configured as a bone punch, so that the distal end 54 of the shaft 14 is moved against a bearing surface 56 when the swiveling handle branch 18 is activated (see FIG. 1), while the surgical instrument 10′ is configured as a conchotome, in which the shaft 14 activates an element 58 of a distal end of the surgical instrument 10′ in the form of a forceps or clamp when the swiveling handle branch 18 is activated (see FIGS. 4A and 4E).

The surgical instrument 10 additionally has spreader springs 24, which hold the handle branches 16 and 18 in the spread apart position; if desired, these can also be provided on the surgical instrument 10′.

For the swivel mounting of the swiveling handle branch 18 on the base part 12, the base part 12 has an opening 24, which lies in particular in the swivel plane. The swiveling handle branch 18 engages by a head 26, which in particular has less material thickness than the rest of the swiveling handle branch 18, through this opening 24, so that it can engage in particular with the working part 20 on the other side of the opening 24. A pivot pin 28 is provided in the region of the opening 24 and engages in a through opening 30, which passes through the swiveling handle branch 18, in order to mount this pivot pin 28 pivotably on the base part 12.

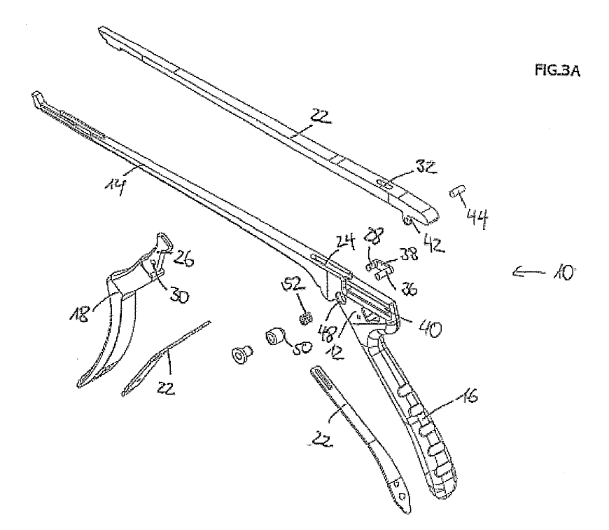

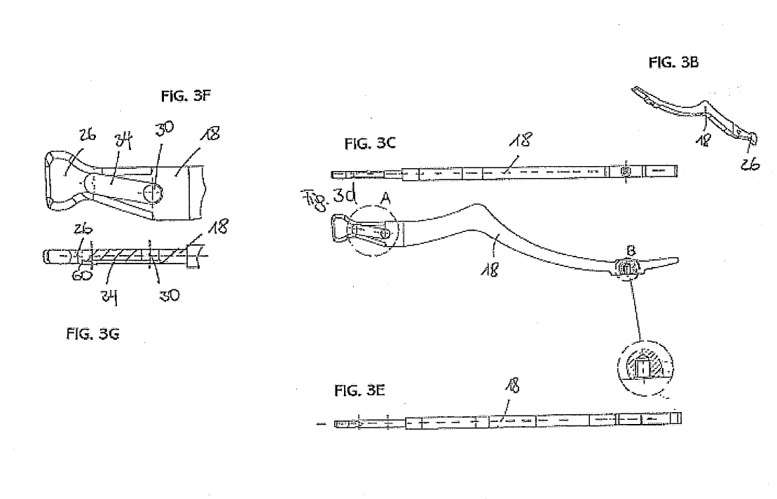

The head 26 is rounded in shape and engages in a recess 32 of the working part 20. The recess 32 is formed in particular as a step bearing for the head 26, so that when the head 26 is shoved into the recess 30 the swiveling handle branch 18 and the working part 20 are positioned relative to each other. When the swiveling handle branch 18 is moved, the head 26 of the swiveling handle branch 18 drives along the working part 20 via the recess 32, so that when the swiveling handle branch 18 is moved against the fixed handle branch 16 one achieves on the whole an axial displacement of the working part 20 relative to the shaft 14 of the base part 12. As is especially evident from FIGS. 3A and 3B, the recess 32 in the surgical instrument 10 is fashioned as an opening through the working part 20. A recess fashioned as an opening is especially easy to clean. Furthermore, the working part 20 can be configured especially flat, in order to allow the user a good view across the working part 20 of the distal end of the surgical instrument 10, 10′.

The mounting of the swiveling handle branch 18 by means of the pivot pin 28 on the base part 12 and the possibility of releasing this pivot connection are especially evident from FIGS. 4C and 4D, but also FIG. 1B.

The axial length of the pivot pin 28 is such that it can engage from one side of the base part 12 in the opening 24 and preferably also come to bear against the opposite side of the opening 24, as shown in FIG. 4C. In particular, the opposite side of the opening 24 can have a correspondingly shaped recess, in which the free end of the pivot pin can engage for a stable mounting of the pivot pin 28.

The pivot pin 28 is joined firmly to a release bolt 36 by a yoke 38 extending essentially perpendicular to its axis. The release bolt 36 runs axially parallel to the pivot pin 28 and is moved by the yoke 38 so far toward the pivot pin 28 and distanced from it that the release bolt 36 is situated outside the swivel range of the head 26 of the swiveling handle branch 13 in the base part 12. The axial length of the release bolt 36 is dimensioned so that the release bolt 36 reaches through the entire base part 12 and sticks out from the base part 12 at the opposite surface. A recess 46 is preferably provided in the outer surface of the base part 12 which can receive the yoke 38 flush with the surface, as long as the release bolt 36 is not depressed, so as not to form any unwanted edges during the use of the surgical instrument.

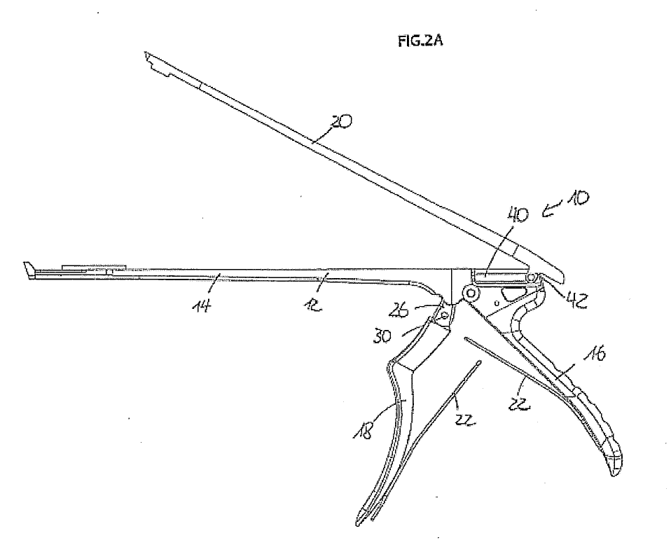

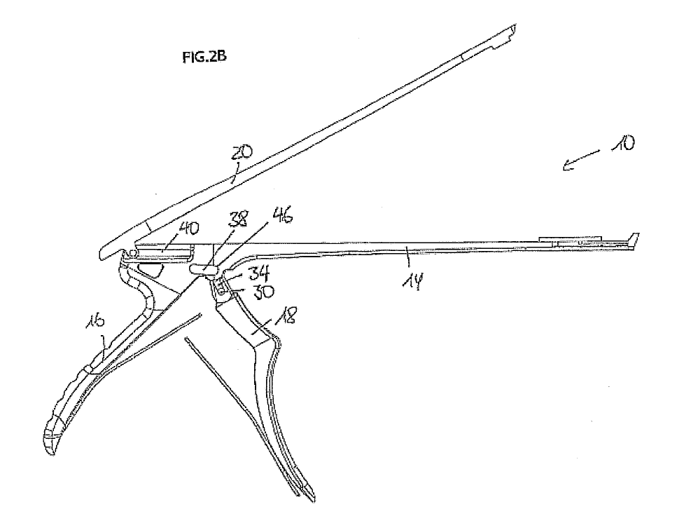

The release bolt 36 can move axially in a borehole 48 of the base part 12. A helical spring 52 is inserted between an outer flange 50 of the release bolt 36 and an inner shoulder of the bore 48, pre-tensioning the release bolt 36 and, with it, the pivot pin 28 in the locking position shown in FIG. 4C. If the release bolt 36 is depressed against the force of the helical spring 52 into the base part 12, the pivot pin 28 is pulled out from the opening 24 of the base part 12 by the yoke 38. This unlocked position is shown in FIG. 4D. In this unlocked position, it therefore becomes possible to pull the swiveling handle branch 18 downward and out from the opening 24 and bring the head 26 out of engagement with the working part 20. In this position, it is therefore possible to detach the working part 20 from the shaft 4 for cleaning purposes, as is shown for example in FIG. 2A, 2B, or 4E.

Basically, it is possible to remove the working part 20 entirely from the base part 12. As shown in FIGS. 2A, 2B, and 4E, the working part 20 still remains connected to the base part 12 by a swiveling connection. In the conchotome of FIG. 4E, this swiveling connection is formed by the activation connection between the shaft and the distal end of the surgical instrument 10′, in the form of a forceps or clamping end. In the surgical instrument 10, per FIGS. 2A and 2B, this swiveling connection is formed by an oblong hole 40 arranged in the base part 12, running in the lengthwise direction of the shaft 14 and passing through the base part 12 transversely to the lengthwise axis of the shaft 14, to which the working part 20 is secured.

The securing is done such that two cheeks 42 are disposed on the working element 20, parallel to and spaced apart from each other, which basically lie against the lateral surfaces of the base part 12 and are joined together by means of a pin 44 running transversely to the cheeks 42. The pin 44 passes through the oblong hole 40, so as to form in this way the swiveling connection between the base part 12 and the working part 20. The base part 12 is configured with reduced width, especially in the region above the oblong hole 14, so that the cheeks 42 do not increase the width of the surgical instrument 10. During the relative movement of the working part 20 relative to the shaft 14, the working part 20 is securely guided by the pin 44 in the oblong hole 40. The guidance is especially robust and stable and in particular requires no special lubrication. If the swiveling handle branch 18 after being unlocked is pulled downward, the head 26 no longer engages in the recess 32 of the working part 20, so that a loosening of the working part 20 from the shaft 12 is made possible. This loosening is done, in particular, by displacing the working part 20 so that a T-shaped element situated in the distal region of the working part 20 can be brought out of engagement with a corresponding T-shaped groove and then the working part 20 can be swiveled about the pin 44 from the shaft 14, so that a cleaning of the region between the working part 20 and the shaft 14 is possible in easy fashion.

In order to hold the swiveling handle branch 18 in this cleaning position and also captively on the base part 12, a groove 34 is disposed in one of the lateral surfaces of the swiveling handle branch 18, starting from the through opening 30, in which the free end of the pivot pin 28 engages after the swiveling handle branch 18 is moved to the unlocked position and pulled slightly out from the base part 12. The groove 34 of the instrument 10 is especially recognizable in FIGS. 2B, 3D, 3F and 3G, while the groove 34 of the instrument 10′ is shown especially in FIG. 4). In FIGS. 4A, 4B and 4M, the groove 34 is indicated by broken lines, but not directly identifiable in these views; instead, it is made in the respective rear side of the handle branch in these views, similar to the swiveling handle branch 18 of the surgical instrument 10 per FIG. 3G.

The groove 34 forms a guideway for the pivot pin 28 during the movement of the swiveling handle branch 18 while pulling the swiveling handle branch 18 out from the base part 12. Furthermore, the end 60 of the groove 34 away from the through opening 30 forms an end stop for the pivot pin 28 during this movement, so that the swiveling handle branch 18 cannot be pulled further out from the base part 12 and is held captively on the base part 12 in this position. At the same time, however, the swiveling handle branch 18 is pulled so far out from the base part 12 that the head 26 is separated from the working part 20 and thus out of engagement with the working part 20, so that the working part can be loosened from the base part 12 without hindrance, as described above. In this way, an especially simple captive securing of the swiveling handle branch 18 to the base part 12 is made possible and in particular the working part 20 is held on the base part 12 independently of the swiveling handle branch 18.

In an alternative embodiment of the invention, not shown, in place of the groove 34 or also in addition to the groove 34 a second through opening is disposed in the swiveling handle branch 18, which is situated, when the groove 34 is present, at the end of the groove 34 away from the through opening 30, so that when the swiveling handle branch 18 is pulled out from the base part 12 the pivot pin 28 in the position separated from the working part engages in the second through opening 30 and thus enables an especially reliable fixation of the swiveling handle branch 18 on the base part 12.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

1-10. (canceled)

11. A surgical instrument, comprising:
(a) a base part having a shaft and a stationary handle branch extending therefrom;
(b) a working part is guided movably on said shaft and a swiveling handle branch is operatively arranged on said base part;
(c) said swivel handle being mounted swivelably in an opening defined in said base part in order to engageably activate said working part by means of a pivot pin;
(d) said pivot pin engages with a through opening in said swiveling handle branch;
(e) said pivot pin, for the purpose of separating said swiveling handle branch from said working part, is entirely operably separable axially from said swiveling handle branch and captively secured to said base part; and
(f) said swiveling handle branch being captively secured to said base part when operatively separated from said working part.

12. A surgical instrument according to claim 11, wherein a groove is arranged on a lateral surface of said swiveling handle branch, starting from a first through opening, with which said pivot pin engages in the position of said swiveling handle branch separated from said working part.

13. A surgical instrument according to claim 11, wherein a second through opening is arranged in said swiveling handle branch, with which said pivot pin engages in the position of said swiveling handle branch separated from said working part.

14. A surgical instrument according to claim 13, wherein said first through opening and said second through opening are joined by means of said groove on said lateral surface of said swiveling handle branch.

15. A surgical instrument according to claim 11, wherein said pivot pin is connected via a yoke to a release bolt, which is arranged axially movable in said base part outside the swivel range of said swiveling handle branch.

16. A surgical instrument according to claim 1, wherein said swiveling handle branch is engaged by a rounded head in a recess of said working part.

17. A surgical instrument according to claim 16, wherein said recess of said working part is formed as an opening through said working part.

18. A surgical instrument according to claim 1, wherein said working part is arranged as to be able to swivel on said base part in the position of said swiveling handle branch separated from said working part.

19. A surgical instrument according to claim 18, wherein said base part has an oblong throughhole passing through a proximal end of said base part and said working part is guided movably in said oblong throughhole and mounted so that it can swivel.

20. A surgical instrument according to claim 19, wherein said base part has a smaller width in the region adjoining said oblong throughhole and on said working part there are arranged a plurality of cheeks extending laterally on said base part, which are joined to a pin that passes through said oblong aperture.

21. A surgical instrument, comprising:
(a) a base part, said base part comprising:
(i) a shaft; and
(ii) a stationary handle branch extending therefrom;
(b) a working part, wherein said working part:
(i) is guided movably on said shaft and a swiveling handle branch is operatively arranged on said base part; and
(ii) is arranged so as to be able to swivel on said base part in the position of said swiveling handle branch separated from said working part;
(c) said swivel handle being mounted swivelably in an opening defined in said base part in order to engageably activate said working part by means of a pivot pin;
(d) said pivot pin engages with a through opening in said swiveling handle branch;
(e) said pivot pin, for the purpose of separating said swiveling handle branch from said working part, is entirely operably separable axially from said swiveling handle branch and captively secured to said base part; and
(f) said swiveling handle branch being captively secured to said base part when operatively separated from said working part.

22. A surgical instrument according to claim 11, wherein a groove is arranged on a lateral surface of said swiveling handle branch, starting from a first through opening, with which said pivot pin engages in the position of said swiveling handle branch separated from said working part.

23. A surgical instrument according to claim 11, wherein a second through opening is arranged in said swiveling handle branch, with which said pivot pin engages in the position of said swiveling handle branch separated from said working part.

24. A surgical instrument according to claim 13, wherein said first through opening and said second through opening are joined by means of said groove on said lateral surface of said swiveling handle branch.

25. A surgical instrument according to claim 11, wherein said pivot pin is connected via a yoke to a release bolt, which is arranged axially movable in said base part outside the swivel range of said swiveling handle branch.

26. A surgical instrument, comprising:
(a) a base part, said base part comprising:
(i) a shaft;
(ii) a stationary handle branch extending therefrom; and
(iii) a proximal end, wherein an oblong throughhole passes through said proximal end;
(b) a working part, wherein said working part:
(i) is guided movably on said shaft and a swiveling handle branch is operatively arranged on said base part;
(ii) is arranged so as to be able to swivel on said base part in the position of said swiveling handle branch separated from said working part; and
(iii) is guided movably in said oblong throughhole and mounted so that said working part can swivel;
(c) said swivel handle being mounted swivelably in an opening defined in said base part in order to engageably activate said working part by means of a pivot pin;
(d) said pivot pin engages with a first through opening in said swiveling handle branch;
(e) said pivot pin, for the purpose of separating said swiveling handle branch from said working part, is entirely operably separable axially from said swiveling handle branch and captively secured to said base part; and
(f) said swiveling handle branch being captively secured to said base part when operatively separated from said working part.

27. A surgical instrument according to claim 26, wherein said base part has a smaller width in the region adjoining said oblong throughhole and on said working part there are arranged a plurality of cheeks extending laterally on said base part, which are joined to a pin that passes through said oblong aperture.

28. A surgical instrument according to claim 26, wherein a groove is arranged on a lateral surface of said swiveling handle branch, starting from a first through opening, with which said pivot pin engages in the position of said swiveling handle branch separated from said working part.

29. A surgical instrument according to claim 28, wherein a second through opening is arranged in said swiveling handle branch, with which said pivot pin engages in the position of said swiveling handle branch separated from said working part, and wherein said first through opening and said second through opening are joined by means of said groove on said lateral surface of said swiveling handle branch.

30. A surgical instrument according to claim 26, wherein said pivot pin is connected via a yoke to a release bolt, which is arranged axially movable in said base part outside the swivel range of said swiveling handle branch.
